(19) United States
(12) Patent Application Publication
(10) Pub. No.: US 2001/0011146 A1
(43) Pub. Date: Aug. 2, 2001

(54) NEUROPROTECTIVE COMPOUNDS AND USES THEREOF

(76) Inventors: TONG H. JOH, NEW YORK, NY (US); SUNGHEE CHO, SCARSDALE, NY (US); CHUNG K. CHU, ATHENS, GA (US); JINFA DU, ATHENS, GA (US)

Correspondence Address: MICHAEL L GOLDMAN, NIXON PEABODY, CLINTON SQUARE, PO BOX 1051, ROCHESTER, NY, 14603

(21) Appl. No.: 09/065,282

(22) Filed: Apr. 23, 1998

Related U.S. Application Data
(60) Provisional application No. 60/044,180, filed on Apr. 23, 1997.

(30) Foreign Application Priority Data
Apr. 23, 1997 (US) ZZZZZZZZZZZZZZ

(51) Int. Cl. C07C 233/00

(52) U.S. Cl. 564/218; 564/219; 564/223; 514/625

(57) ABSTRACT

The present invention relates to a compound having the formula:

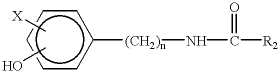

where

X=R₁O, F, Br, I, Cl, or a C₁ to C₅ alkyl group,

R₁=a C₁ to C₁₀ alkyl group or a C₁ to C₁₀ aryl group,

n=1 or 2,

R₂=a C₁ to C₆ alkyl group, an amino acid, a heterocycle, a secondary or tertiary C₃ to C₄ hydrocarbon, or

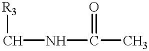

where

R₃=H or CH₃,

or pharmaceutically-acceptable salts thereof. The invention further relates to pharmaceutical compositions which include the compounds, as well as methods of making and using the compounds.

The present application claims priority benefit of U.S. Provisional Patent Application Ser. No 60/044,180, filed Apr. 23, 1997, which is hereby incorporated by reference.

### FIELD OF THE INVENTION

The present invention relates to neuroprotective compounds and their use in treating patients suffering from neural cell degeneration, death, or disorder.

### BACKGROUND OF THE INVENTION

Certain neurons in the mammalian central nervous system are more vulnerable to ischemia than others (Pulsinelli, W. A., “Selective Neuronal Vulnerability: Morphological and Molecular Characteristics,” Prog. Brain Res., 63:29-37 (1985)). While excessive release of the excitatory amino acids and activation of their receptors have been suggested to be the cause of neuronal death (Benveniste, et al., “Elevation of the Extracellular Concentrations of Glutamate and Aspartate in Rat Hippocampus During Transient Cerebral Ischemia Monitored by Intracerebral Microdialysis,” J. Neurochem., 43:729-34 (1984) and Olney, J. W., “Excitotoxicity and N-methyl-D Aspartate Receptors,” Drug Dev. Res., 17:299-319 (1989)), oxygen-derived radicals are also implicated in the neuronal injury that occurs following ischemia/reperfusion (Kirsch, J. R., et al., “Evidence for Free Radical Mechanisms of Brain Injury Resulting from Ischemia/Reperfusion-Induced Events,” J. Neurotrauma, 9(Suppl. 1):S157-63 (1992); Traystman, R. J., et al., “Oxygen Radical Mechanisms of Brain Injury Following Ischemia and Reperfusion,” J. Appl. Physiol., 71:1185-95 (1991); and IIalliwell, B., “Reactive Oxygen Species and the Central Nervous Systems,” J. Neurochem., 59:1609-23 (1992)). Accordingly, various free radical scavengers/antioxidants have been administered in ischemic models and shown to protect neurons in the central nervous system (Umemura, K., et al., “Effect of 21 -aminosteroid Lipid Peroxidation Inhibitor, U74006F, in the Rat Middle Cerebral Artery Occlusion Model,” European J. Pharmacol., 251:69-74 (1994); Sutherland, G., et al., “Ischemic Neocortical Protection with U74006F—A Dose-Response Curve,” Neurosci. Lett., 149:123-25 (1993); Sorrenti, V., et al., “Lipid Peroxidation and Survival in Rats Following Cerebral Post-Ischemic Reperfusion: Effect of Drugs With Different Molecular Mechanisms,” Drug Under Exp. Clin. Res., 20:185-89 (1994)). In addition, transgenic animals over-expressing human superoxide dismutase (SOD-1) have shown to be resistant to ischemic/reperfusion injury (Chan, P. H., et al., “SOD-1 Transgenic Mice as a Model for Studies of Neuroprotection in Stroke and Brain Trauma,” Annals New York Acad. Sci., 738:93-103 (1994) and Yang, G., et al., “Human Copper-Zinc Superoxide Dismutase Transgenic Mice are Highly Resistant to Reperfusion Injury After Focal Cerebral Ischemia,” Stroke, 25:165-70 (1994)). However, treatment modalities with free radical scavengers have been greatly hindered due to their inability to penetrate the blood drain barrier.

Melatonin is a neurohormone secreted from pineal gland. In vitro studies showed that melatonin acts as a free radical scavenger (Manev, H., et al., “In Vitro and In Vivo Protection With Melatonin Against the Toxicity of Singlet Oxygen,” Nerosci. Abstr., 21:1518 (1995); Longoni, B., et al., “Melatonin Inhibits Oxygen Radicals Induced Lipid Damage,” Neurosci Abstr., 21:485 (1995); Reiter, R. J., et al., “A Review of the Evidence Supporting Melatonin's Role as an Antioxidant,” J. Pineal Res., 18:1-11 (1995); and Reiter, R. J., “Oxidative Processes and Antioxidative Defense Mechanisms in the Aging Brain,” FASAB J., 9:526-33 (1995)) and readily penetrates into the central nervous system after peripheral administration (Reiter, R. J., Melatonin: That Ubiquitously Acting Pineal Hormone,” News Physiol. Sci., 6:223-27 (1991)). Recently, it was demonstrated that the administrative of melatonin during cerebral reperfusion protects the CA1 hippocampal neurons after 10 minutes of transient forebrain ischemia (Cho, S., et al., “Melatonin Administration Protects CA 1 Hippocampal Neurons After Transient Forebrain Ischemia in Rats.” Brain Research, 755:335-38 (1997)), perhaps via its antioxidant property. However, a delay of one to two hours in administration significantly decreased protection of the neurons (Id.). In addition, melatonin is insoluble in aqueous media, hence, it is difficult lo prepare and administer to humans.

The present invention is directed toward overcoming these deficiencies.

### SUMMARY OF INVENTION

The present invention relates to a compound having the formula:

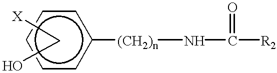

where

X=R₁O, F, Br, I, Cl, or a C₁ to C₅ alkyl group,

R₁=a C₁ to C₁₀ alkyl group or a C₁₀ to C₁₀ aryl group,

n=1 or 2,

R₂=a C₁ to C₆ alkyl group, an amino acid, a heterocycle, a secondary or tertiary C₃ to C₄ hydrocarbon, or

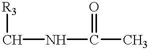

where

R₃=H or CH₃, or pharmaceutically-acceptable salts thereof.

The compounds of the present invention can be used to treat patients having a neural degenerative disease which includes administering to the patient the compound under conditions effective to treat the neural degenerative disease. The compounds can be used to treat patients suffering from Alzheimer's Disease, Parkinson's Disease, aging, stroke, multiple sclerosis, neurotrauma, and amyotrophic lateral sclerosis.

Further, the compounds can be used in a method of preventing cell death or degeneration by providing the compound to a neuronal cell under conditions effective to prevent cell death or degeneration.

In addition, the compounds are useful in methods of inhibiting the activity of Interleukin 1 β converting enzyme, nitric oxide synthase, or GTP cyclohydrolase I in a neuron by contacting the neuron with the compound.

The present invention also relates to a method of producing the compound.

The compound of the present invention can be used to treat diseases and disorders which are related to neuronal degeneration, disorder, or death. The compound of the present invention is water soluble, allowing for intravenous administration. Further, the compound of the present invention is more potent than melatonin in its neuroprotective capacity.

### BRIEF DESCRIPTION OF THE DRAWINGS

FIG. 1 shows a mean neuronal density of the CA1 hippocampus of male Wister rats after 10 minutes of ischemia. CA1 hippocampal neurons in all three treatment groups are significantly protected compared to the saline treated group. Most protection, however, is seen in the group whose treatments are started immediately after reperfusion (45% of sham operated control group).

FIGS. 2A-D show NADPH-diaphorase histochemistry in control hippocampus. The figures show the presence of intensely stained NADPH-diaphorase positive neurons in CA1 (FIG. 2B), but not in other pyramidal (FIGS. 2C and 2D) and granular cell (FIG. 2E)) layers.

FIGS. 3A-H show a temporal profile of NADPH-diaphorase histochemistry in postischemic hippocampus. NADPH-diaphorase staining is shown in control (FIG. 3A), 12 hour (FIG. 3B), 24 hour (FIG. 3C), 2 days (FIG. 3D), 3 days (FIG. 3E), and 7 days (FIG. 3F) after 0 minutes of four-vessel occlusion ischemia. The presence of intense staining in CA1 region of hippocampus after ischemia was greatest after 24 hours of ischemia. High magnification of CA1 neurons after 24 hours of ischemia indicates the presence of staining in the cytoplasm of pyramidal neurons (FIG. 3G). The presence and absence of staining is clear at the junction of CA1/2 (FIG. 31H). The arrow indicates the junction of CA1 and CA2.

FIGS. 4A-D show NADPH-diaphorase staining in CA1 hippocampus in untreated (saline) and treated ischemic animals. NADPH-diaphorase staining is darker in saline treated CA1 hippocampus at 24 hours (FIG. 4A) and 48 hours (FIG. 4C) compared to neuroprotective compound treated CA1 hippocampus at 24 hours (FIG. 4B) and 48 hours (FIG. 4D).

FIG. 5 shows nitrite levels in BV-2 microglia cells. Treatment with lipopolysaccharide (“LPS”) increased nitrite levels. The addition of the compound of the present invention reduced nitrite levels in a dose-dependent manner.

FIG. 6 shows the total number of BV-2-cells in 24 well plates. No difference of cell number was noted regardless of the presence of LPS and the compound of the present invention.

FIGS. 7A-C show NADPH-diaphorase histochemical staining in BV-2 cells. NADPH-diaphorase staining was performed in the absence of LPS (FIG. 7A), the presence of LPS (FIG. 7B), the presence of LPS and 5 mM compound of the present invention (FIG. 7C). The marked increase in staining in the presence of LPS (FIG. 7B) was attenuated by treatment with the compound of the present invention (FIG. 7C).

### DETAILED DESCRIPTION OF THE INVENTION

The present invention relates to a compound having the formula:

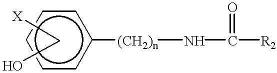

where

X=R₁O, F, Br, I, Cl, or a C₁ to C₅ alkyl group,

R₁=a C₁ to C₁₀ alkyl group or a C₁ to C₁₀ aryl group,

n=1 or 2,

R₂=a C₁ to C₆ alkyl group, an amino acid, a heterocycle, a secondary or tertiary C₃ to C₄ hydrocarbon, or

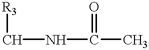

where

R₃=H or CH₃,

or pharmaceutically-acceptable salts thereof.

One preferred compound includes where X is R₁O, particularly where R₁ a methyl group, where R₂ is a C₁ to C₆ alkyl group, particularly a methyl group, and where n is 2. Another preferred compound is where X is R₁O and R₂ is

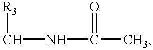

where R₃ and R₁ are methyl groups, and n is 2.

This invention also includes pharmaceutically acceptable salts in the form of inorganic or organic acid or base addition salts of the above compounds. Suitable inorganic acids are, for example, hydrochloric, hydrobromic, sulfuric, and phosphoric acids. Suitable organic acids include carboxylic acids, such as, acetic, propionic, glycolic, lactic, pyruvic, malonic, succinic, fumaric, malic, tartaric, citric, cyclamic, ascorbic, maleic, hydroxymaleic, dihydroxymaleic, benzoic, phenylacetic, 4-aminobenzoic, anthranilic, cinnamic, salicylic, 4-aminosalicylic, 2-phenoxybenzoic, 2-acetoxybenzoic, and mandelic acid. Sulfonic acids, such as, methanesulfonic, ethanesulfonic, and β-hydroxyethane-sulfonic acid are also suitable acids. Non-toxic salts of the compounds of the above-identified formulas formed with inorganic and organic bases include, for example, those alkali metals, such as, sodium, potassium, and lithium, alkaline earth metals, for example, calcium and magnesium, light metals of group IIIA, for example, aluminum, organic amines, such as, primary, secondary, or tertiary amines, for example, cyclohexylamine, ethylamine, pyridine, methylaminoethanol, and piperazine. These salts, are prepared by conventional means, for example, by treating the compounds of the present invention with an appropriate acid or base.

Treating neural cells with one or more of the compounds of the present invention inhibits degeneration of the cells leading to cell death. Furthermore, these compounds when administered to a patient are effective to inhibit various neural degenerative diseases in patients suffering from these diseases.

As used herein, the term “neural degenerative disease” refers to those diseases in mammals, including humans, in which symptoms are due to degeneration, death, or trauma of nerve cells (i.e., neurons of any type and bodily location, including the brain, the central nervous system, and the periphery). This degeneration, death, or trauma is thought to be caused by damage inflicted by oxygen-derived free radicals. Explicitly included within the term “neural degenerative disease” are aging, stroke, Alzheimer's Disease, Parkinson's Disease, multiple sclerosis (“MS”), amyotrophic lateral sclerosis (“ALS”), or neurotrauma. This list is exemplary, not exclusive. The method described herein can be used to treat other neural degenerative diseases in addition to those disorders listed.

The compounds herein may be made up in any suitable form appropriate for the desired use; e.g., oral, parenteral (for example, subcutaneously, intravenously, intramuscularly, intraperitoneally, by intranasal instillation, by application to mucous membranes, such as that of the nose, throat, and bronchial tubes, or by instillation into hollow organ walls or newly vascularized blood vessels), or topical administration. Suitable dosage forms for oral use include tablets, dispersible powders, granules, capsules, suspens ons, syrups, and elixirs. The compounds may be administered alone or with suitable pharmaceutical diluents or carriers. Inert diluents and carriers for tablets include, for example, calcium carbonate, sodium carbonate, lactose, and talc. Tablets may also contain granulating and disintegrating agents such as starch and alginic acid, binding agents such as starch, gelatin, and acacia, and lubricating agents such as magnesium stearate, stearic acid, and talc. Tablets may be uncoated or may be coated by known techniques to delay disintegration and absorption. Inert diluents and carriers which may be used in capsules include, for example, calcium carbonate, calcium phosphate, and kaolin. Suspensions, syrups, and elixirs may contain conventional excipients, for example, methyl cellulose, tragacanth, sodium alginate; wetting agents, such as lecithin and polyoxyethylene stearate; and preservatives, e.g. ethyl-p-hydroxybenzoate.

Dosage forms suitable for parenteral administration include solutions, suspensions, dispersions, emulsions, and the like. They may also be manufactured in the form of sterile solid compositions which can be dissolved or suspended in sterile injectable medium immediately before use. They may contain suspending or dispersing agents known in the art. Such agents include sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions.

It will be appreciated that the actual preferred amount of the compound to be administered according to the present invention will vary according to the particular compound, the particular composition formulated, and the mode of administration. Many factors that may modify the action of the inhibitor can be taken into account by those skilled in the art; e.g., gender, body weight, diet, time of administration, route of administration, rate of excretion, condition of the subject, drug combinations, and reaction sensitivities and severities. Administration can be carried out continuously or periodically within the maximum tolerated dose. Optimal administration rates for a given set of conditions can be ascertained by those skilled in the art using conventional dosage administration tests.

In particular, the quantity of the compound administered may vary over a wide range to provide in a unit dosage an effective amount of from about 0.1 to 10 mg/kg of body weight of the patient per day to achieve the desired effect.

The compounds of the present invention possess anti-degenerative activity in neural cells and can be used in the treatment of stroke (i.e., apoplexy). After the initial onset of stroke, progressive and further injury to the neurons deprived of oxygen can occur during the intense respiratory burst which occurs as the acute blockage is cleared (normally with anti-coagulant treatment such as heparin or coumarin). This respiratory burst generates oxygen-derived free radical species which cause further damage to the already weakened neurons.

The compounds preferably are administered as soon as possible after the onset of stroke to prevent ischemic or reperfusion injury as the thrombosis or embolism subsides and normal circulation is restored to the effected area. Preferably, the treatment is begun well within 24 hours of onset of the stroke.

The invention thus provides a method of treating stroke in a patient afflicted with stroke comprising administering to the patient one or more compounds of the present invention in an amount effective to inhibit stroke-related neural degeneration.

Alzheimer's disease is characterized by the presence of senile plaques in the brain. While the etiology of Alzheimer's disease is unknown, the plaques are thought to be due to free radical damage, which leads to cell death and the formation of the plaques. Consequently, by treating brain cells with compounds of the present invention, via administration of the compounds to an Alzheimer's patient in need thereof, damage to the patient's brain cells can be inhibited.

The subject invention thus provides a method of treating Alzheimer's disease in a patient afflicted with Alzheimer's disease which comprises administering to the patient a compound of the present invention in an amount effective to inhibit progression of the Alzheimer's disease.

Multiple sclerosis (“MS”) is another neural degenerative disorder where free radicals inflict cellular damage to neurons. It is also of unknown etiology.

Experimental Allergic Encephalomyelitis (“EAE”), an animal model for multiple sclerosis, is mediated by immune mechanisms in which macrophage activation and the generation of oxygen-derived free radicals play a major role. In mice, induced EAE causes reversible paralysis which mimics multiple sclerosis. Left untreated, induced EAE normally resolves spontaneously approximately 8 to 10 days after the onset of symptoms.

The invention thus provides a method of treating multiple sclerosis in a patient afflicted with multiple sclerosis comprising administering to the patient a compound of the present invention in an amount effective to inhibit progression of the multiple sclerosis.

Amyotrophic lateral sclerosis (“ALS”) is related to multiple sclerosis in that its symptoms are caused by sclerotic degeneration of the spinal cord leading to progressive muscular atrophy. Its etiology is also unknown.

The invention thus provides a method of treating amyotrophic lateral sclerosis in a patient afflicted with amyotrophic lateral sclerosis which comprises administering to the patient a compound of the present invention in an amount effective to inhibit progression of the amyotrophic lateral sclerosis.

In particular, the compounds of the present invention inhibit the activity of interleukin 1 β converting enzyme, nitric oxide synthase, and/or GTP cyclohydrolase I, thereby preventing neuronal death, degeneration, or trauma. Interleukin 1 β converting enzyme (“ICE”) activity is associated with apoptosis and ICE inhibitors play an important role as antiapoptic drugs which specifically inhibit ICE activity to prevent apoptotic cell death. Nitric oxide synthase (“NOS”) activity produces the nitric oxide radical NO, which plays an important role in cell death and degeneration. GTP-cyclohyrolase I in an enzyme important in the production of BH₄, which is required in the production of NO. Thus, inhibition of the activity of these, or other, enzymes prevents or neuronal cell death, degeneration, and trauma.

The compounds of the present invention can be used to treat warm blooded animals, such as mammals. Examples of such beings include humans, cats, dogs, horses, sheep, cows, pigs, lambs, rats, mice, and guinea pigs.

The compounds of the present invention are prepared by reacting a compound having the formula:

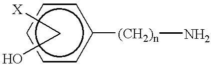

where X is R₁O, F, Br, I, Cl, or a C₁ to C₅ alkyl group, and R₁ is a C₁ to C₁₀ alkyl group or a C₁ to C₁₀ aryl group with an acyl compound having the formula:

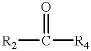

where R₄ is a leaving group known to one of ordinary skill in the art, such as a halide or an acetate, and where R₂ is a C₁ to C₆ alkyl group, an amino acid, a hetereocycle, a secondary or tertiary C₃ to C₄ hydrocarbon, or

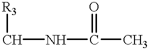

where R₃ is H or CH₃ under conditions effective to produce a compound having the formula:

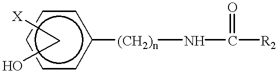

Preferably, the acyl compound is an acid anhydride or an acid halide having a leaving group well known to those of ordinary skill in the art.

More preferably, the acyl compound is an acid anhydride having the formula:

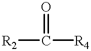

where R₄ is

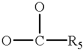

where R₅ is an alkyl or an aryl.

The reaction is carried out in a solvent, such as chloroform, methylene chlorides or acetonitrile, with methylene chloride being especially preferred. The reaction is carried out for a period of from about 0.5 to about 6 hours, at a temperature of from about 0° to about 80° C., and at a pressure of from about 1 to about 2 atmospheres.

### EXAMPLES

To understand the role of nitric oxide (“NO”) in ischemic neuronal injury, it was investigated whether ischemia alters nicotinamide adenine dinucleotide phosphate (“NADPH”)-diaphorase activities differentially in selectively vulnerable CA1 neuron. Using one neuroprotective agent of the present invention, additional investigation was done to determine if NADPH-diaphorase activity in CA1 hippocampus of N-acetyl-3-O-methyldopamine (“NAMDA”)-treated animals differs from that of saline-treated animals, and if it does, whether the alteration of NADPH-diaphorase activities are correlated with neuroprotection. To establish if the effect of the compound of the present invention in vivo is mediated via the nitric oxide synthase (“NOS”) system, a microglial cell line was used that express iNOS in the presence of lipopolysaccharide (“LPS”), to determine whether treating the cells with the compound affected nitrite (the oxidation product of NO) accumulation and NADPH-diaphorase activity.

Materials and Methods

Synthesis of N-acetyl-3-O-methyldopamine (“NAMDA”). 3-O-methyldopamine hydrochloride (1 g, 4.9 mmol) (Aldrich Chemical Company, Milwaukee, Wis.) was suspended in 10 ml of methytlene chloride and 2 ml of triethylamine. Acetyl anhydride (1 g, 9.8 mmol) was added and the solution was refluxed for 3 hours. After refluxing, the solvent was removed in vacuo and the residue was redissolved in 10 ml of methanol. Next, 200 mg of potassium carbonate was added to the solution and the resulting mixture was stirred at room temperature for 3 hours. Methanol was removed and the residue was purified by silica gel column chromatography (0-5% of methanol in chloroform) to give N-acetyl-3-O-methyldopamine (930 mg, 91%) as a semi-syrup, which on standing solidified in several weeks. The chemical structure of the synthesized compound was identified by spectroscopic analyses: NMR (DMSO-_{d6}) δ8.71 (s, 1H, OH, D₂O exchangeable), 7.87 (br t, J=4.8, 1H, NH, D₂O exchangeable), 6 73 (d, J=1.6 Hz, 1H, 2-H), 6.67 (d, J=8 Hz, 1H, 5-H), 6.57 (dd, J=1.6, 8Hz, 6-H), 3.74 (s, 3H, OCH₃), 3.18, 2.57 (q,t, J=7.6, 7.2Hz, 4H, CH₂CH₂), 1.78 (s, 3H, Ac). Anal. Calcd. for C₁₁H₁₅NO₃H₂O: C, 60.66; H, 7.02; N, 6.36. Found: C, 60.56; H, 7.04; N, 6.29. MS m/z; 210 [M+H]⁺.

Four-vessel occlusion (“4-VO”) ischemia. All procedures regarding animals were in compliance with AALAC guidelines set forth in the PHS animal “Guide in the Care and Use of Laboratory Animals”. Animals (male Wister rats, 200-250 gr, Hill Top, Scottsdale) were anesthetized with a mixture of halothane (1%), oxygen, and nitrogen, and surgically prepared for 4-VO according to the method described by Pulsinell, W. A., et al., “Regional Cerebral Blood Flow and Glucose Metabolism Following Transient Forebrain Ischemia,” Ann. Neurol., 11:499-502 (1982), which is hereby incorporated by reference. Surgical procedures included placing reversible clasps around the common carotid arteries and placing a suture around the neck muscles to control collateral blood flow to the brain. Food was withheld overnight, but water was freely available. On the following day, 10 minutes of 4-VO ischemia was induced by tightening the clasps around the common carotid arteries and the suture. In order to minimize variability, the following criteria was set: loss of righting reflex and bilateral pupil dilation during the entire ischemic period, and 20±5 minutes of postischemic coma after 10 minutes of ischemia. Only animals that meet these criteria were included in the study. The body temperature of all animals was kept at 37.5±0.5° C. by a thermocouple-regulated heating lamp during ischemia and reperfusion until the animals regained consciousness and established thermo-homeostasis.

NAMDA administration. Animals subject to 10 minutes of ischemia randomly were divided into 4 groups. Animals received one of the following triple intraperitoneal injections: i) saline at 0, 0.5, and 2 hours, ii) NAMDA (10 mg/kg) at 0, 0.5, and 2 hours, iii) NAMDA at 1, 1.5, and 3 hours, and iv) NAMDA at 2, 2.5, and 4 hours of cerebral reperfusion. To examine whether NAMDA caused hypothermia, the animals' body temperatures were recorded for up to the first 4 hours of cerebral reperfusion. Sham-operated animals that underwent surgery and carotid manipulation were used as non-ischemic controls.

Tissue preparation. Animals were anesthetized with sodium pentobarbital (120 mg/kg) and perfused transcardially with saline containing 0.5% sodium nitrite and 10 U/ml heparin sulfate followed by 4% cold formaldehyde in 0.1 M sodium phosphate buffer (PB, pH 7.2). The brains were further postfixed for 2 hours and stored in a 30% sucrose solution overnight. Fixed brains were sectioned at 30 μm on a sliding microtome. For each animal, the dorsal hippocampus between bregma −2.5 mm and −4.0 mm was sampled. Some sections were counted on slides and stained with cresyl violet to measure neuronal density. Others were used for free floating NADPH-diaphorase histochemistry.

Cell density measurement. An unbiased morphometric strategy was used to measure neuronal density in the CA1 region of hippocampus (Cho, S., et al., “Melatonin Administration Protects CA1 Hippocampal Neurons After Transient Forebrain Ischemia in Rats,” Brain Res., 755:335-38 (1997), which is hereby incorporated by reference). Briefly, a 100×100 μm frame (10 boxes on a side) was placed so that its vertical axis was perpendicular to the stratum pyramidale, and then this frame was systematically passed along the entire length of the CA1 region. The CA1-CA2 border was identified by the change in neuron shape and packing density. All sections were viewed under oil with a 1.2 N.A. lens. The counting frame was a 50 μm×100 μm subsection of the frame. Neurons were counted in the frame if part or all of the nucleus was within the frame and not in contact with the left or bottom border of the frame. For each animal, neurons in the right and left stratum pyramidale were sampled from comparable regions of the anterior dorsal hippocampus (bregma −3.2 mm) and the posterior dorsal hippocampus (bregma −3.8 mm). Four sections at least 300 μm apart were obtained for each anima. The number of neurons counted were divided by the total volume sampled to generate the density of neurons in CA1. Mean neuron density was calculated for the left and right hippocampus (side) and for the anterior and posterior regions for each animal. Neuron density was analyzed in a three factor (treatment, region, and side) ANOVA followed by post-hoc testing (Fisher's PLSD).

NADPH-Diaphorase histochemistry. The histochemical staining was performed according to the method described by Vincent, et al., “Histochemical Mapping of Nitric Oxide Synthase in the Rat Brain,” Neuroscience, 46:755-784 (1992), which is hereby incorporated by reference). Sections containing dorsal hippocampus are washed twice in 0.1 M phosphate buffer (“PB”) and then processed for NADPH-diaphorase histochemistry. To establish a temporal profile of NADPH-diaphorase staining during postischemic period, sections were obtained from animals that were perfuse fixed at 12 hours, 24 hours, 48 hours, 72 hours, and 7 days after ischemia. The sections were then incubated for 1 hour at 37° C. with a solution containing 1 mg/ml of NADPH, 0.25 mg/ml of nitro blue tetrazolium (“NBT”), and 0.3% Triton X-100 in 0.1 M PB. The reaction was terminated by the addition of cold 0.1M PB. Sections were mounted on slides, dehydrated, coverslipped, and examined under a light microscope.

Nitrite measurement on microylial cell. To measure nitrite level, a NO oxidative metabolite, murine BV-2 cells, were used. The cell line has been shown to exhibit phenotypic and functional properties of reactive microglial cells (Blasi, et al. “Immortalization of Murine Microglia Cells By a v-raf/v-myc Carrying Retrovirus,” J. Neuroimmunology, 27:229-237 (1990), which is hereby incorporated by reference). The cells were grown and maintained in Dulbeccos Modified Eagle medium (“DMEM”, Gibco, Gaithersburg, Md.) supplemented with 10% fetal calf serum and penicillin/streptomycine at 37° C. in a humidified incubator under 5% CO₂. BV-2 microglia cells were cultured and grown in 24 well culture plates and treated for 6 hours with 0. 0.05, 0.5, 2, or 5 mM or NAMDA either in the presence or absence of lipopolysaccharide (LPS, 0.2 μg/ml).

Accumulated nitrite amount was measured in the cell supernatant by the Griess reaction (Green, et al., “Analysis of Nitrate, Nitrite, and ¹⁵N nitrate in Biological Fluids,” Anal. Biochem., 126:131-138 (1982), which is hereby incorporated by reference). After the treatment, 200 μl aliquots of cell supernatant from each well were mixed with 100 μl of Griess reagent (1 % sulphanilamide, 0.1% naphthylethylenediamine dihydrochloride, 2.5% H₃PO₄) in 96 well microtiter plates. The mixtures were incubated for 10 minutes to form a chromophore and the absorbance was read at 540 nm using a plate reader. The amount of nitrite accumulation from media was determined against a standard curve generated by a known concentration of NaNO₃. After removal of the supernatant for the nitrite assay, cells were immediately washed with 0.1M PB, fixed with 4% formaldehyde for 30 minutes, and washed with 0.1M PB for 5 minutes. NADPH-diaphorase histochemical staining was performed as described above. An exact duplicate of 24 wells in the presence and absence of LPS were used to count the number of cells by tryphan exclusion method after treatment with various concentrations of NAMDA.

### Example 1

### Neuroprotection by NAMDA

The animals' body temperature was kept at 37.5±0.5° C. during ischemia and first half hour of cerebral reperfusion when animals were typically stayed in postischemic coma. Temperatures were recorded soon after animals regained consciousness and recorded for up to 4 hours of cerebral reperfusion (Table 1).

TABLE 1
Temperature Recordings of Control and NAMDA Treated
Ischemic Animals During Cerebral Reperfusion

| Postischemic | | | NC-111 | NC-111 |
|---|---|---|---|---|
| time | Control | NC-111 | (1 h) | (2 hr) |
| (hour) | (n = 12) | (n = 9) | (n = 7) | (n = 6) |
| 0.5 | 37.9 ± 0.2 | 37.7 ± 0.2 | 37.9 ± 0.2 | 38.3 ± 0.2 |
| 1 | 37.2 ± 0.1 | 37.6 ± 0.2 | 36.9 ± 0.1 | 36.8 ± 0.3 |
| 1.5 | 37.4 ± 0.1 | 37.3 ± 0.1 | 37.0 ± 0.1 | 37.2 ± 0.4 |
| 2 | 37.5 ± 0.2 | 37.3 ± 0.1 | 37.4 ± 0.1 | 37.4 ± 0.2 |
| 3 | 37.6 ± 0.2 | 37.5 ± 0.2 | 37.5 ± 0.2 | 37.3 ± 0.2 |
| 4 | 37.8 ± 0.2 | 37.2 ± 0.1 | 37.5 ± 0.3 | 37.9 ± 0.3 |

Data are expressed as mean ± s.e.m.

There were no differences in body temperatures between the saline-treated ischemic and NAMDA-treated ischemic groups at any time points recorded (ANOVA, Newman-Keuls Multiple Comparison Test). This data suggest that administration of NAMDA does not affect animal's body temperature during and for a few hours after the treatments.

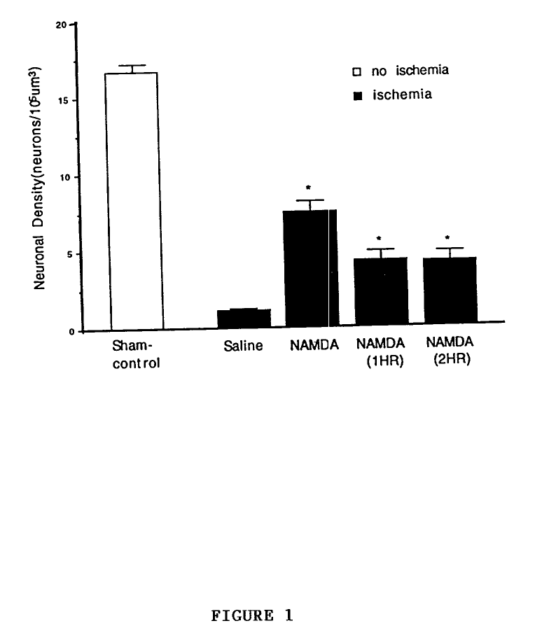

Neuronal density was measured one week later. There was no significant interaction among treatment, region, and side. Ischemia induced by 4-VO lead to significant decrease of neuronal density aid treatment of NAMDA significantly protected neurons in CA1 hippocampus (FIG. 1, Fisher's PLSD, p<0.0001). Although most protection was achieved in the animal group that received NAMDA treatment immediately after reperfusion (45% of non-ischemic control), delaying administration of the drug up to 2 hours after ischemia also resulted in significant protection of CA1 neurons against ischemia.

The duration of ischemia may determine the temporal profile and fate of cell death. To investigate whether 10 minutes of ischemia causes early cell death (less than 24 hours) as well as delayed neuronal death (a few days after ischemia) and to examine which type of cell death will be prevented by NAMDA treatment, neuron density was measured in CA1 at 24 hours of postischemic time point in saline- and NAMDA-treated animals and then compared with non-ischemic sham controls. No difference was found among three groups (sham-controls (n=7), 16.9±2.7 neurons/10⁵ μm³; saline-ischemic (n=3), 15.9±2.9 neurons/10⁵ μM³ NAMDA-ischemic (n=3), 18.3±2.2 neurons/10⁵ μm³). The data indicate that 10 minutes of ischemia does not cause any detectable early necrotic death and that the CA1 neurons that were protected by NAMDA treatment (FIG. 1) are the population of neurons that would otherwise undergo delayed cell death.

### Example 2

### NADPH-diaphorase activity in vivo

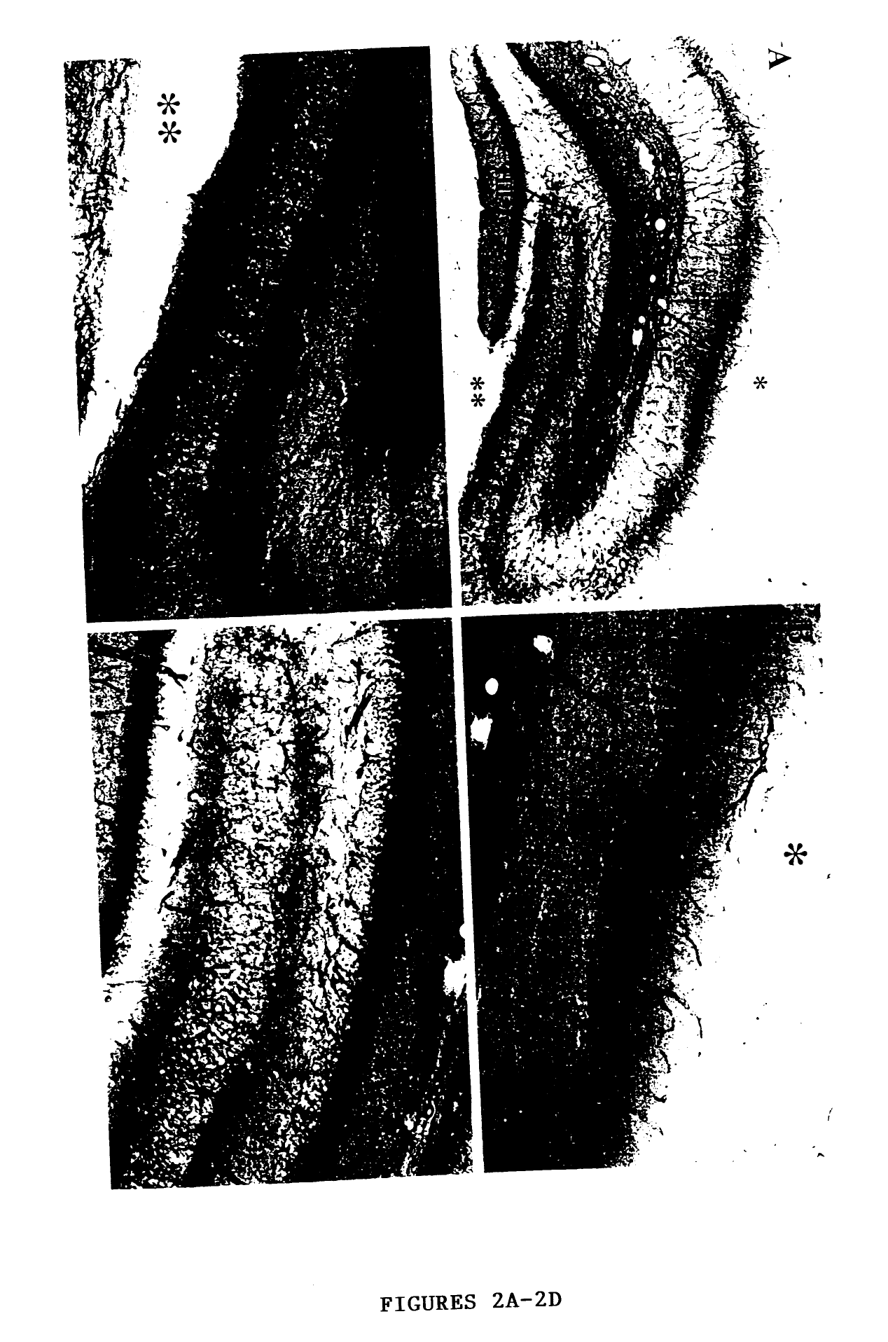

To investigate NO involvement in selective neuronal injury, the presence of NADPH-diaphorase positive neurons in control brain was examined. Intensely stained NADPH-diaphorase positive neurons are scattered in CA1 pyramidal layers (FIGS. 2A and 2B). These neurons are very few or mostly absent in CA2-4 pyramidal layers (FIGS. 2C and 2D). In dentate gyrus, intensely NADPH-diaphorase staining neurons are located adjacent to but not in the granular cell layer (FIG. 2D). These observations suggest that the physical location of NADPH-diaphorase positive neurons in CA1 hippocampus may contribute to selective neuronal vulnerability, perhaps acting as a major source of NO and killing neighboring pyramidal neurons during postischemic period.

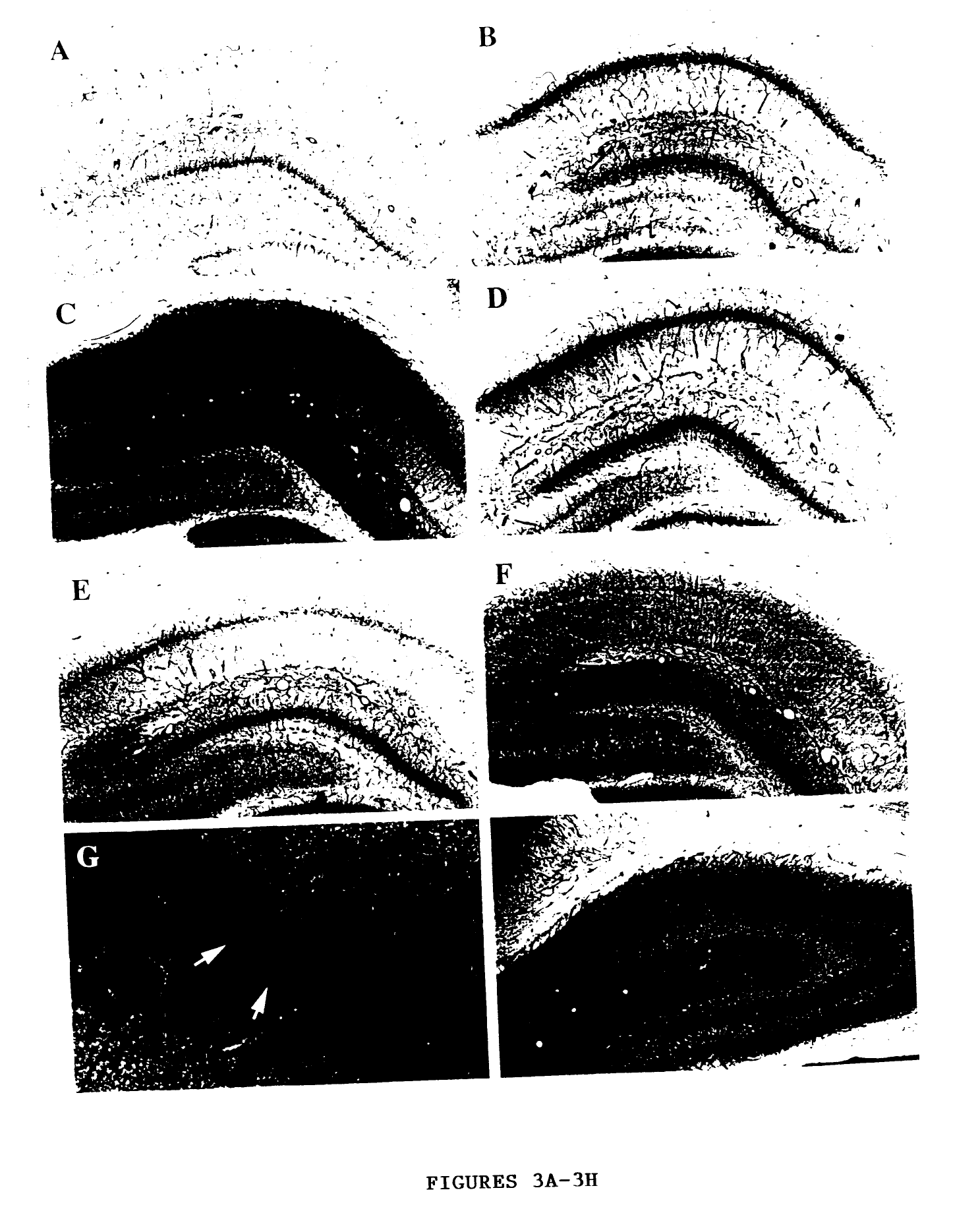

Next, investigation was done to determine if ischemia alters NADPH-diaphorase activity in CA1 pyramidal neurons. Compared to sham-operated controls. 10 minutes of 4-VO ischemia induced NADPH-diaphorase activity in selectively vulnerable CA1 neurons. The intensity of staining was significantly elevated at 12 hours, peaked at 24 hours, and reduced at 3 days after ischemia (FIGS. 3B-3E). The lack of the staining at 7 days after ischemia may be due to CA1 cell loss (FIG. 3F). Ischemia-induced NADPH-diaphorase staining is specifically localized in the cytoplasm of CA1 pyramidal neurons (FIG. 3G). The presence and absence of ischemia-induced NADPH-diaphorase activity was demarcated at the junction of CA1/2 pyramidal neurons (FIG. 3H, see arrow). Although some degree of NADPH-diaphorase staining was present in the regions adjacent to CA2-4 pyramidal and dentate granula cell layers, CA2-4 pyramidal neurons and granular neurons in dentate gyrus were devoid of staining. The data indicated the NADPH-diaphorase activity in CA1 pyramidal neurons is up-regulated by ischemia and the up-regulation is a region-specific.

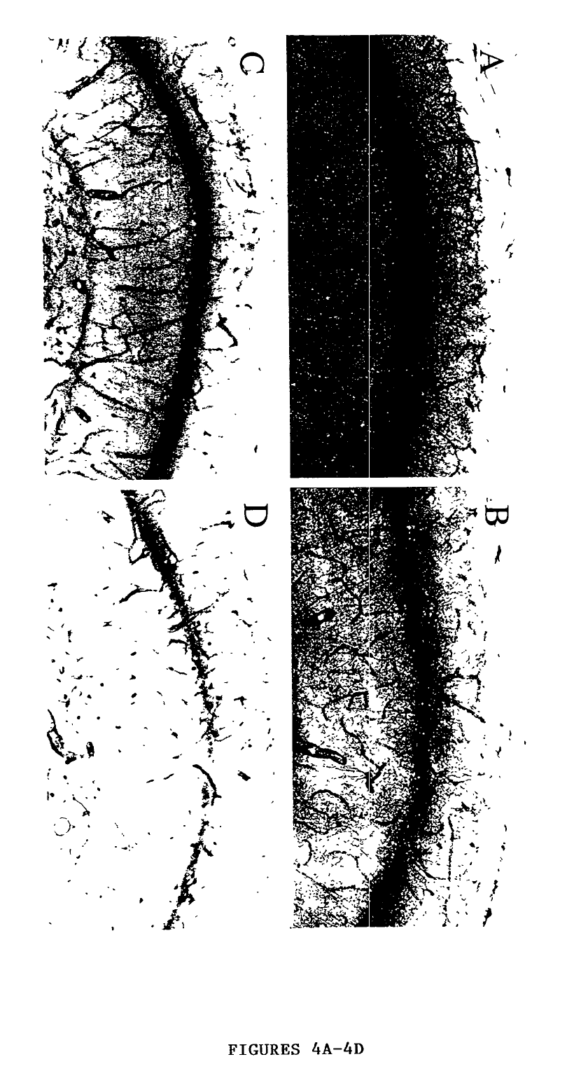

To investigate whether neuroprotective action of NAMDA is mediated through the alteration of NOS activity, NADPH-diaphorase staining in saline- and NAMDA-treated animals was performed during postischemic period. Ischemia-induced NADPH-diaphorase staining at 24 hours of postischemic time point was markedly reduced by triple intraperitoneal injection of NAMDA (10 mg/kg) during reperfusion (FIGS. 4A and 4B). The attenuation of the staining was persisted 48 hours after ischemia (FIGS. 4C and 4D). The same treatment protected 45% of CA1 pyramidal neurons from 10 minutes of ischemia (FIG. 1). Taken together, the in vivo data indicate that regionally up-regulated NADPH-diaphorase activity in pyramidal neurons by ischemia may play an important role in selective neuronal injury and that the attenuation of NADPH-diaphorase activity in CA1 pyramidal neurons during reperfusion may account for the neuroprotection achieved by NAMDA treatment.

### Example 3

### NADPH-diaphorase activity and nitrite levels in vitro

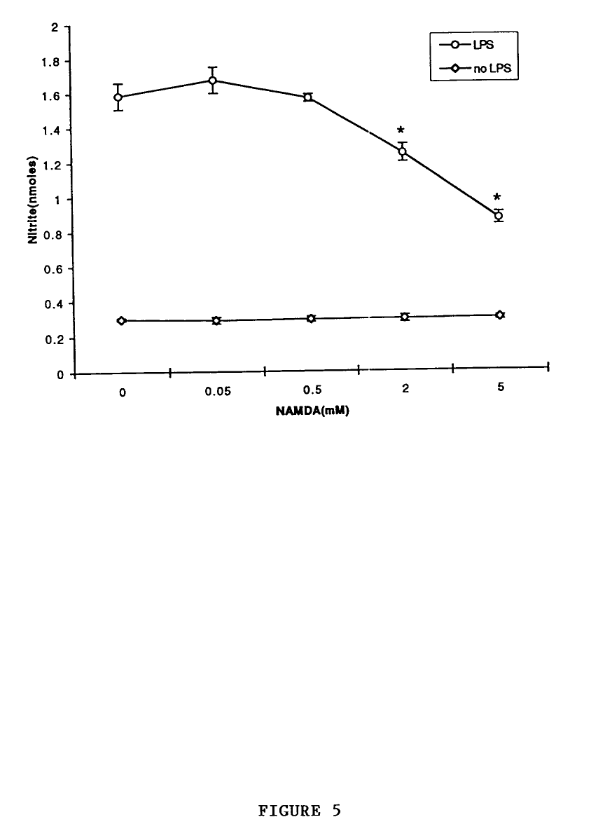

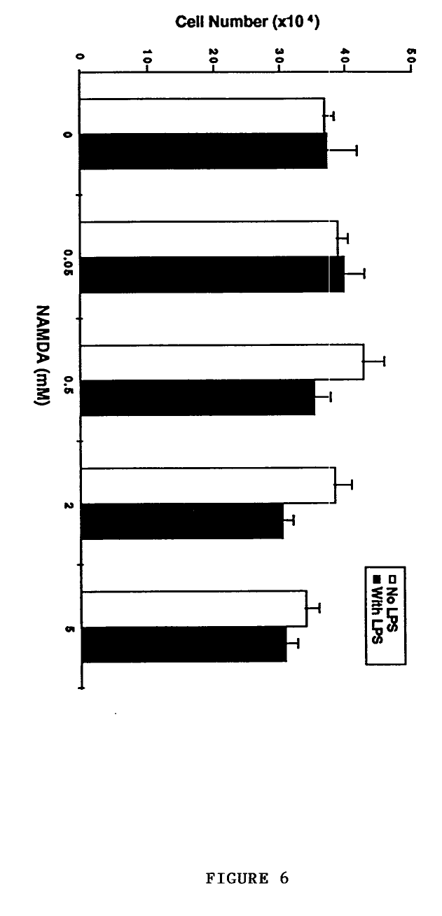

To establish whether the neuroprotective effect of NAMDA observed in vivo could be mediated via inhibition of NADH-diaphorase activity of NOS and subsequent reduction of NO generation during post-ischemic period, NADPH-diaphorase activity and nitrite levels (an oxidation product of NO), in BV-2 cells was determined. There was low but measurable nitrite accumulation in the supernatant of the cells in the absence of LPS (FIG. 5). The addition of NAMDA, however, did not alter nitrite accumulation. On the other hand, the treatment with LPS increased the nitrite level 5-6 times compared to control. Moreover, the addition NAMDA significantly reduced nitrite accumulation in a dose-dependent manner (FIG. 5, ANOVA, p<0.001, Neuman-Kuels multiple comparison). To investigate whether high concentrations of NAMDA affected cell viability, cell number was counted at the end of treatment. NAMDA treatment did not affect the total number of cells, regardless of the presence of LPS (FIG. 6). Taken together, the data indicate that NAMDA treatment reduces LPS-stimulated NO generation without affecting cell viability.

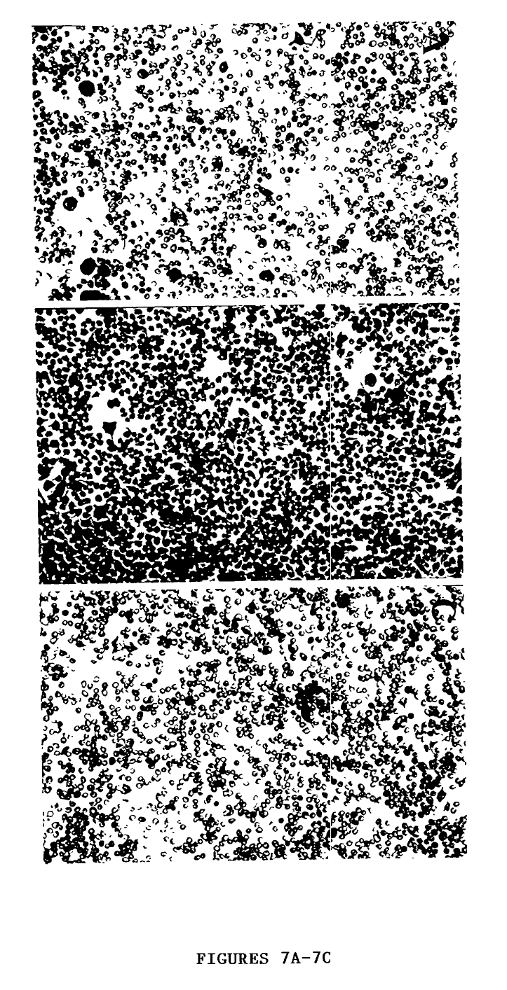

To investigate whether the reduction in nitrite levels in the BV-2 cells is associated with NADPH catalytic activity of NOS, NADPH-diaphorase histochemical staining was performed in the cells after removal of supernatant and fixation. In the absence of LPS, there was little NADPH-diaphorase staining (FIG. 7A) and the baseline intensity of staining was not affected by 5 mM of NAMDA treatment (data not shown). In contrast, treatment with LPS produced an increase in NADPH-diaphorase activity (FIG. 7B) that was attenuated by 5 mM NAMDA treatment (FIG. 7C). The NADPH-diaphorase histochemical staining is in agreement with the biochemical (nitrite level) data, indicating that the neuroprotective action of NAMDA observed in vivo is likely to be mediated via the reduction of NOS catalytic activity and subsequent attenuation of NO generation during postischemic reperfusion.

Discussion

A brief episode of transient forebrain ischemia causes selective neuronal death in the CA1 hippocampus in experimental animals models and in humans (Pulsinelli, W. A., et al., “A New Model of Bilateral Hemispheric Ischemia in the Unanesthetized Rat,” Stroke, 10:267-72 (1973); Kirino, T., “Delayed Neuronal Death in the Gerbil Hippocampus Following Ischemia,” Brain Res., 239:57-69 (1982); Petito, C., “Delayed Hippocampal Damage in Human Following Cardiorespiratory Arrest,” Neurology, 37:1281-86 (1987), which are hereby incorporated by reference). NO, synthesized from L-arginine by the enzyme NOS, is a free radical that acts as a signaling molecule in normal synaptic transmission. It has been shown that NO biosynthesis is profoundly altered in pathologic condition, and considerable evidence suggests NO is involved in the pathophysiology of cerebral isehemia (Iadecola, C., “Bright and Dark Sides of Nitric Oxide in Ischemia Brain Injury,” Trends Neurosci., 20:132-39 (1997), which is hereby incorporated by reference). Increased NO generation and upregulated NOS mRNA and protein have been reported in experimental isehemic animal models (Kader, A., et al., “Nitric Oxide Production During Focal Cerebral Ischemia in Rats,” Stroke, 24:1709-16 (1993); Zhang, et al., “Upregulation of Neuronal Nitric Oxide Synthase and mRNA, and Selective Sparing of Nitric Oxide Synthase-Containing Neurons After Focal Cerebral Ischemia In Rat,” Brain Res., 654:85-95 (1994) (“Zhang”); Iadecola, C., et al.. “Marked Induction of Calcium-Independent Nitric Oxide Synthase Activity After Focal Cerebral Ischemia,” J. Cereb. Blood Flow & Metab., 15:52-59 (1995); and Iadecola, C., et al., “Inducible Nitric Oxide Synthase Gene Expression in Brain Following Cerebral Ischemia.” J. Cereb. Blood Flow & Metab., 15:378-84 (1995). which are hereby incorporated by reference). NOS containing neurons and NOS catalytic activity are determined by NADPH-diaphorase histochemical staining (Dawson, T. M., et al., “Nitric Oxide Synthase ard Neuronal NADPH Diaphorase are Identical in Brain and Peripheral Tissues,” Proc. Natl. Acad. Sci. USA, 88:7797-7801 (1991) and Hope, B. T., et al., “Neuronal NADPH Diaphorase is a Nitric Oxide Synthase,” Proc. Natl. Acad. Sci. USA, 88:2811-14 (1991), which are hereby incorporated by reference).

The data indicate that altering NADPH-diaphorase activity, may play a role in neuroprotection. After occluding the middle cerebral artery, increased NO production is accompanied by the up-regulation of nNOS gene and protein (Kader, A., et al., “Nitric Oxide Production During Focal Cerebral Ischemia in Rats.” Stroke, 24:1709-16 (1993) and Zhang, which are hereby incorporated by reference), up-regulated protein and activity of eNOS (Nagafuji, T., et al., “Temporal Profiles of Ca2+/calmodulin-dependent and—independent Nitric Oxide Synthase Activity in the Rat Brain Microvessels Following Cerebral Ischemia,” Acta Neurochirupica, 60(suppl.):285-88 (1994) and Zhang, which are hereby incorporated by reference) and of iNOS (Iadecola, C., et al., “Inducible Nitric Oxide Synthase Gene Expression in Brain Following Cerebral Ischemia.” J. Cereb. Blood Flow & Metab., 15:378-84 (1995) and Iadecola, C., et al., “Inducible Nitric Oxide Synthase Gene Expression in Vascular Cells After Transient Focal Cerebral Ischemia,” Stroke, 27:1373-80 (1996), which are hereby incorporated by reference) indicating the NO/NOS involvement in ischemic neuronal injury. However, the results obtained from treatment of NOS inhibitors in vivo is quite controversial (Nagafuji T., et al., “Blockade of Nitric Oxide Formation by N-omega-nitro-L-arginine Mitigates Ischemic Brain Edema and Subsequent Cerebral Infarction in Rats,” Neurosci. Lett., 147:159-62 (1992); Buisson, A., et al., “The Neuroprotective Effect of a Nitric Oxide Inhibitor in a Rat Model of Focal Cerebral Ischaemia.” Br. J. Pharmacol., 106:766-67 (1992); Kohno, K., et al., “Intraventricular Administration of Nitric Oxide Synthase Inhibitors Prevents Delayed Neuronal Death in Gerbil Hippocampal CA1 Neurons,” Neurosci. Lett., 199:65-68 (1995); Izumi, Y., et al., “Nitric Oxide Inhibitors Attenuate N-methyl-D-aspartate Excitotoxicity in Rat Hippocampal Slices,” Neurosci. Lett., 135:227-30 (1992); Izumi. Y., et al., “Nitric Oxide Inhibitors Attenuate Ischemic Degeneration in the CA1 Region of Rat Hippocampal Slices,” Neurosci. Lett., 210:157-60 (1996); Shapiro, et al., “Dose-Dependent Effect of Nitric Oxide Synthase Inhibitor Following Transient Forebrain Ischemia In Gerbils,” Brain Res., 668:80-84 (1994); Hamada, Y., et al., “Inhibitor of Nitric Oxide Synthesis Reduces Hypoxic-Ischemic Brain Damage in the Neonatal Rat,” Pediatr. Res., 35:10-14 (1994), which are hereby incorporated by reference). More conclusive results came from mice with targeted disruption of nNOS. eNOS, or iNOS genes. When nNOS or iNOS null mice were subjected to focal ischemia, there was a reduction of infarct size (Huang, Z., et al., “Effects of Cerebral Ischemia in Mice Deficient in Neuronal Nitric Oxide Synthase,” Science, 265:1883-85 (1994) and Iadecola, C., et al., “Delayed Reduction of Ischemic Brain Injury and Neurological Deficits in Mice Lacking the Inducible Nitric Oxide Synthase Gene,” J. Neurosci., 17:9157-64 (1997), which are hereby incorporated by reference) and the attenuation of CA1 damage in nNOS mutant (Panahian, N., et al., “Attenuated Hippocampal Damage After Global Cerebral Ischemia in Mice Mutant in Neuronal Nitric Oxide Synthase,” Neurosci., 72:343-54 (1996), which is hereby incorporated by reference). Further study showed that infarct size of nNOS mutant became larger after a treatment with a selective eNOS inhibitor, nitro-L-arginine (Huang, Z., et al., “Effects of Cerebral Ischemia in Mice Deficient in Neuronal Nitric Oxide Synthase.” Science, 265:1883-85 (1994), which is hereby incorporated by reference). These results support the view that NO produced by nNOS or iNOS appears to potentiate ischemic injury, although their actions might be temporally distinct, while NOS produced by eNOS protects against ischemic injury. Thus, the present findings of ischemia-induced NADPH-diaphorase activity and the attenuation of NAMDA in CA1 hippocampus suggest either nNOS or iNOS involvement in selective neuronal injury and that NAMDA may act as a NOS inhibitor.

In a focal ischemic model, the expression of nNOS occurs shortly after the induction of ischemia. The increase in nNOS mRNA suggests the possibility of induction of the gene after ischemia (Wu, W., et al., Neuroscience, 61:719-26 (1994), which is hereby incorporated by reference). An increase in NADPH-diaphorase staining in postischemic CA1 hippocampus was observed relatively early (i.e., before cell injury occurs). The intensity of staining was specific and the temporal profile of histochemical staining was comparable with reports by Kato, et al., “Induction of NADPH-diaphorase Activity in the Hippocampus in a Rat Model of Cerebral Ischemia and Ischemic Tolerance,” Brain Res., 652:71-75 (1994), which is hereby incorporated by reference, where they postfixed gerbil brain for 6 hours after 6 minutes of global ischemia. On the other hand, iNOS expression is delayed and temporally separated from nNOS. Compared to the focal ischemia, the progression of CA1 neuronal death after 10 minutes of global isehemia requires at least 4-5 days. Thus, the facts that the induction of NADPH-diaphorase staining is specifically localized in the cytoplasm of the pyramidal neurons (FIG. 3G) and occurs as early as 3 hours (date not shown) after 10 minutes of global ischemia and that iNOS expression is shown to be localized in astrocytes in the same model (Endoh, M., et al., “Reactive Astrocytes Express NADPH-Diaphorase In Vivo After Transient Ischemia,” Neuroscience Lett., 154:125-28 (1993) and Endoh, M., et al. “Expression of the Inducible Form of Nitric Oxide Synthase by Reactive Astrocytes After Transient Global Ischemia,” Brain Res., 651:92-100 (1994), which are hereby incorporated by reference) suggests possible contribution of nNOS to selective neuronal injury.

Recently, it was demonstrated that melatonin administration starting immediately after reperfusion, significantly protect CA1 neurons, but delay of one hour did not offer significant protection (Cho, S., et al., “Melatonin Administration Protects CA1 Hippocampal Neurons After Transient Forebrain Ischemia in Rats,” Brain Res., 755:335-38 (1997), which is hereby incorporated by reference). In addition to melatonin's action as an antioxidant and a free radical scavenger (Reiter, R. J., “Oxygen Radical Detoxification Processes During Aging: The Functional Important of Melatonin,” Aging, 7:340-51 (1995), which is hereby incorporated by reference), melatonin also has other protective effects including inhibiting nitric oxide synthase (Pozo, D., et al., “Physiological Concentrations of Melatonin Inhibit Nitric Oxide Synthase in Rat Cerebellum,” Life Sci., 55:455-60 (1995), which is hereby incorporated by reference), stimulating glutathionie peroxidase (Barlow-Walden, L., et al., “Melatonin Stimulates Brain Glutathione Peroxidase Activity,” Neurochem. Intl., 26:497-502 (1995), which is hereby incorporated by reference), and reducing lipid peroxidation (Melchiorri, D., et al., “Melatonin Reduces Kainate-Induced Lipid Peroxidation in Homogenates of Different Brain Regions,” Fed. Am. Soc. Exp. Biol. J., 9:1205-10 (1995), which is hereby incorporated by reference). It is possible NAMDA may exert its neuroprotective action via one of these mechanisms. However, since NAMDA, but not melatonin, protect CA1 neurons despite delaying the treatment up to 2 hours, the findings suggest possible differential neuroprotective mechanisms afforded by NAMDA, such as acting through the NOS system. Alternatively, NAMDA may modulate exogenous factors such as noradrenergic or serotonergic input to hippocampus that could alter the level of BH4, an essential cofactor for NOS biosynthesis, and indirectly affect the NOS system. NO production by NOS requires an essential cofactor, (6R)-5,6,7,8-tetrahydro-L-biopterin (BH₄) (Kwon, N. S., et al., “Reduced Biopterin as a Cofactor in the Generation of Nitric Oxide by Murine Macrophages”, J. Biol. Chem., 264:20496-20501 (1989) and Gross S. S., et al., “Cytokine-activated Endothelial Cells Express an Isotype of Nitric Oxide Synthase Which is Tetrahydrobiopterin-dependent, Calmoduline-independent and Inhibited by Arginine Analogs With a Rank-order of Potency Characteristic of Activated Macrophages,” Biochem. Biophys. Res. Comm., 178:823-829(1991), which are hereby incorporated by reference). BH₄ is synthesized from GTP via sequential enzyme reactions including GTP-cyclohydrolase (GTPCH, the first and rate limiting enzyme) and two more enzymes. It is assumed teat inhibition of BH₄ production will lead to lowering NO production, and, hence, projects neuronal degeneration after ischemia.

In summary, NAMDA administration during cerebral reperfusion protects CA1 neurons after 10 minutes of transient 4-VO ischemia. Induction of NADPH-diaphorase activity in CA1 pyramidal neurons after ischemia suggests NOS involvement in selective neuronal death in this region. Furthermore, the attenuation of NADPH-diaphorase activity by NAMDA indicates that the neuroprotective action of the drug maybe be mediated via the reduction of NOS activity and subsequent reduction of NO generation, the view supported by biochemical as well as NADPH-diaphorase histochemical data in vitro.

Although the invention has been described in detail for the purpose of illustration, it is understood that such detail is sorely for that purpose, and variations can be made therein by those skilled in the art without departing from the spirit and scope of the invention which is defined by the following claims.

What is claimed:

1. A compound having the formula: 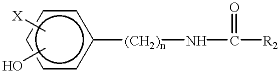
where
X=R₁O, F, Br, I, Cl, or a C₁ to C₅ alkyl group,
R₁=a C₁ to C₁₀ alkyl group or a C₁ to C₁₀ aryl group,
n=1 or 2,
R₂=a C₁ to C₆ alkyl group, an amino acid, a heterocycle, a secondary or tertiary C₃ to C₄ hydrocarbon, or 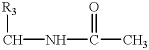
where
R₃=H or CH₃, or pharmaceutically-acceptable salts thereof.

2. The compound according to claim 1, wherein
X is R₁O,
R₂ is a C₁ to C₁₀ alkyl group, and
n=2.

3. The compound according to claim 2, wherein
R₁ and R₂ are methyl groups.

4. The compound according to claim 1, wherein
X is R₁O and
R₂ is

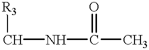

5. The compound according to claim 4, wherein
R₁ and R₃ are methyl groups and n is 2.

6. A pharmaceutical composition comprising:
the compound according to claim 1 and
a pharmaceutically acceptable carrier.

7. A pharmaceutical composition comprising:
the compound according to claim 3 and
a pharmaceutically acceptable carrier.

8. A pharmaceutical composition comprising:
the compound according to claim 5 and
a pharmaceutically acceptable carrier.

9. A method of treating a patient having a neural degenerative disease comprising:
administering to the patient the compound according to claim 1 under conditions effective to treat the neural degenerative disease.

10. The method according to claim 9, wherein:
X is R₁O,
n=2, and
R₁ and R₂ are methyl groups.

11. The method according to claim 9, wherein:
X is R₁O,
R₂ is 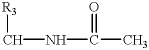
R₁ and R₃ are methyl groups, and
n is 2.

12. The method according to claim 9, wherein the compound is administered orally, parenterally, or topically.

13. The method according to claim 12, wherein the compound is administered intravenously.

14. The method according to claim 13, wherein the neural degenerative disease is selected from the group consisting of Parkinson's Disease, Alzheimer's Disease, aging, stroke, multiple sclerosis, neurotrauma, and amyotrophic lateral sclerosis.

15. A method of preventing neuronal cell death or degeneration comprising:
providing the compound according to claim 1 to a neuronal cell under conditions effective to prevent neuronal cell death or degeneration.

16. The method according to claim 15, wherein:
X is R₁O,
n=2, and
R₁ and R₂ are methyl groups.

17. The method according to claim 15, wherein:
X is R₁O,
R₂ is 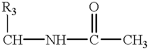
R₁ and R₃ are methyl groups, and
n is 2.

18. The method according to claim 15, wherein the compound is administered orally, parenterally, or topically.

19. The method according to claim 18, wherein the compound is administered intravenously.

20. A method of inhibiting the activity of Interleukin 1 β converting enzyme in a neuron comprising:
contacting the neuron with the compound according to claim 1 under conditions effective to inhibit the activity of Interleukin 1 β converting enzyme.

21. The method according to claim 20, wherein
X is R₁O,
n=2, and
R₁ and R₂ are methyl groups.

22. The method according to claim 20, wherein
X is R₁O,
R₂ is 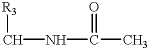
R₁ and R₃ are methyl groups, and
n is 2.

23. A method of inhibiting the activity of nitric oxide synthase in a neuron comprising:
contacting the neuron wits the compound according to claim 1 under conditions effective to inhibit the activity of nitric oxide synthase.

24. The method according to claim 23, wherein
X is R₁O,
n=2,and
R₁ and R₂ are methyl groups.

25. The method according to claim 23, wherein
X is R₁O,
R₂ is 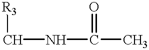
R₁ and R₃ are methyl groups, and
n is 2.

26. A method of inhibiting the activity of GTP cyclohydrolase I in a neuron comprising:
contacting the neuron with the compound according to claim 1 under conditions effective to inhibit the activity of GTP cyclohydrolase I.

27. The method according to claim 26, wherein
X is R₁O,
n=2, and
R₁ and R₂ are methyl groups.

28. The method according to claim 26, wherein
X is R₁O,
R₂ is 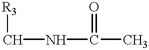
R₁ and R₃ are methyl groups, and
n is 2.

29. A method of producing a neuroprotective compound, said method comprising:
reacting a compound having the formula: 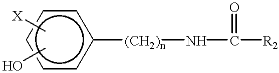
where X is R₁O, F, Br, I, Cl, or a C₁ to C₅ alkyl group, and R₁ is a C₁ to C₁₀ alkyl group or a C₁ to C₁₀ aryl group, with an acyl compound having the formula: 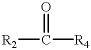
where R₄ is a leaving group, and
R₂ is a C₁ to C₆ alkyl group, an amino acid, a heterocycle, a secondary or tertiary C₃ to C₄ hydrocarbon, or 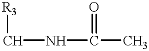
where R₃ is H or CH₃,
under conditions effective to produce the neuroprotective compound having the formula:

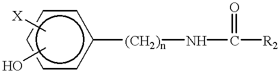

30. The method according to claim 29, wherein
X is R₁O,
R₂ is a C₁ to C₁₀ alkyl group, and
n=2.

31. The method according to claim 30, wherein
R₁ and R₂ and methyl groups.

32. The compound according to claim 29, wherein
X is R₁O and
R₂ is

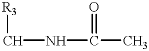

33. The method according to claim 32, wherein R₁ and R₃ are methyl groups.

34. The method according to claim 29, wherein the acyl compound is an acid anhydride or an acid halide.

35. The method according to claim 34, wherein R₄ is 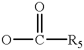
where R₅ is an alkyl or an aryl.
